(19) United States
(12) Patent Application Publication
(10) Pub. No.: US 2025/0268545 A1
(43) Pub. Date: Aug. 28, 2025

(54) PORTABLE MEDICAL DIAGNOSTIC DEVICE

(71) Applicant: Cyrus Beamon, East Lansing, MI (US)

(72) Inventor: Cyrus Beamon, East Lansing, MI (US)

(21) Appl. No.: 18/589,564

(22) Filed: Feb. 28, 2024

(51) Int. Cl. A61B 6/00; A61B 6/10; A61B 6/40; A61B 6/46

(52) U.S. Cl. CPC A61B 6/4417; A61B 6/107; A61B 6/4035; A61B 6/4405; A61B 6/463; A61B 6/5294; A61B 6/54; A61B 6/563

(57) ABSTRACT

A portable medical diagnostic device for conveniently obtaining a temperature, blood pressure, and x-ray image of a patient includes a housing having an interior space. A central processing unit is positioned in the interior space. A display screen is coupled to the housing and is electrically coupled to the central processing unit. The display screen includes a temperature display section, a blood pressure display section, and an x-ray display section. An x-ray scanner electrically coupled to the central processing unit produces an x-ray image of a user for display on the x-ray display section. A temperature scanner electrically coupled to the central processing unit detects a temperature of the user for display on the temperature display section. A blood pressure scanner electrically coupled to the central processing unit detects a blood pressure of the user for display on the blood pressure display section.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to portable medical scanners and more particularly pertains to a new portable medical scanner for conveniently obtaining a temperature, blood pressure, and x-ray image of a patient.

### (2) Description of Related Art Including Information Disclosed Under 37 CFR 1.97 and 1.98

The prior art relates to portable medical scanners. Medical scanners are vital diagnostic tools. Nearly every visit to a doctor's office includes a temperature scan and a blood pressure scan. However, properly diagnosing a patient often also requires an x-ray image. X-ray images are typically obtained using a fixed x-ray machine, which can increase patient wait times as patients queue for use of the machine. Thus, there is a need in the art for a portable x-ray scanner that can also provide a temperature reading and a blood pressure reading. Such a device could help reduce patient wait times. Such a device could also ease the burden on medical staff, who could use a single device to obtain diagnostic information instead of needing to obtain and use multiple scanners.

### BRIEF SUMMARY OF THE INVENTION

An embodiment of the disclosure meets the needs presented above by generally comprising a housing having a top wall, a bottom wall, and a peripheral wall that is coupled to and extends between the top wall and the bottom wall to define an interior space. A central processing unit is positioned in the interior space. A power source is positioned in the interior space and is electrically coupled to the central processing unit. A display screen is coupled to the housing and is electrically coupled to the central processing unit. The display screen is positioned on the top wall. The display screen includes a temperature display section, a blood pressure display section, and an x-ray display section.

An x-ray scanner is coupled to the housing. The x-ray scanner is electrically coupled to the central processing unit and is configured to emit a plurality of x-ray waves outwardly through the bottom wall to produce an x-ray image of a body part of a user. The central processing unit is configured to display the x-ray image on the x-ray display section of the display screen.

A temperature scanner is positioned in the interior space. The temperature scanner is electrically coupled to the central processing unit and is configured to detect a temperature of the user. The central processing unit is configured to display the temperature on the temperature display section of the display screen.

A blood pressure scanner is positioned in the interior space. The blood pressure scanner is electrically coupled to the central processing unit and is configured to detect a blood pressure of the user. The central processing unit is configured to display the blood pressure on the blood pressure display section of the display screen.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of a portable medical diagnostic device according to an embodiment of the disclosure.

FIG. 2 is a front view of an embodiment of the disclosure.

FIG. 3 is a rear view of an embodiment of the disclosure.

FIG. 4 is a side view of an embodiment of the disclosure.

FIG. 5 is a side view of an embodiment of the disclosure.

FIG. 6 is an in-use view of an embodiment of the disclosure.

FIG. 7 is a detail view of an embodiment of the disclosure.

FIG. 8 is a detail view of an embodiment of the disclosure.

### DETAILED DESCRIPTION OF THE INVENTION

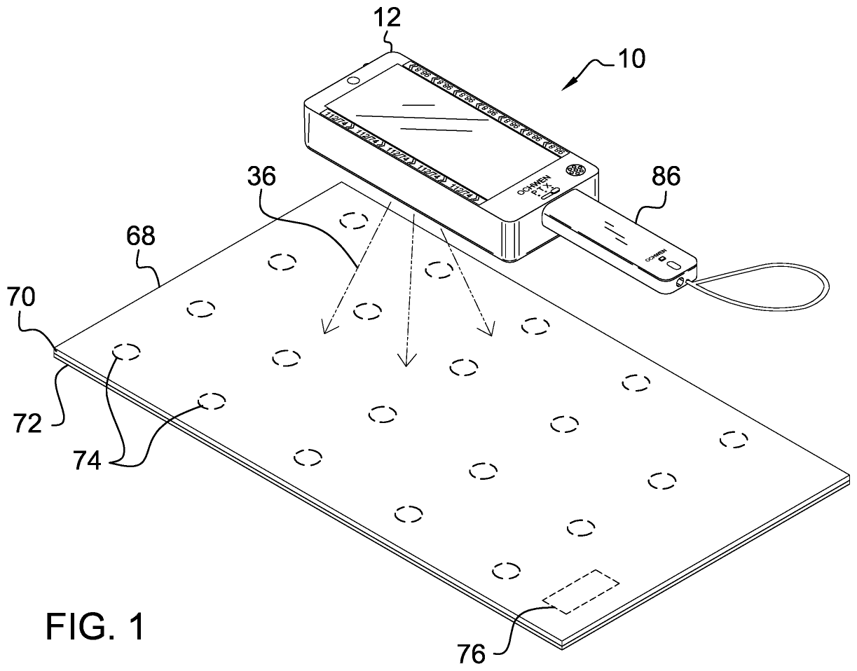

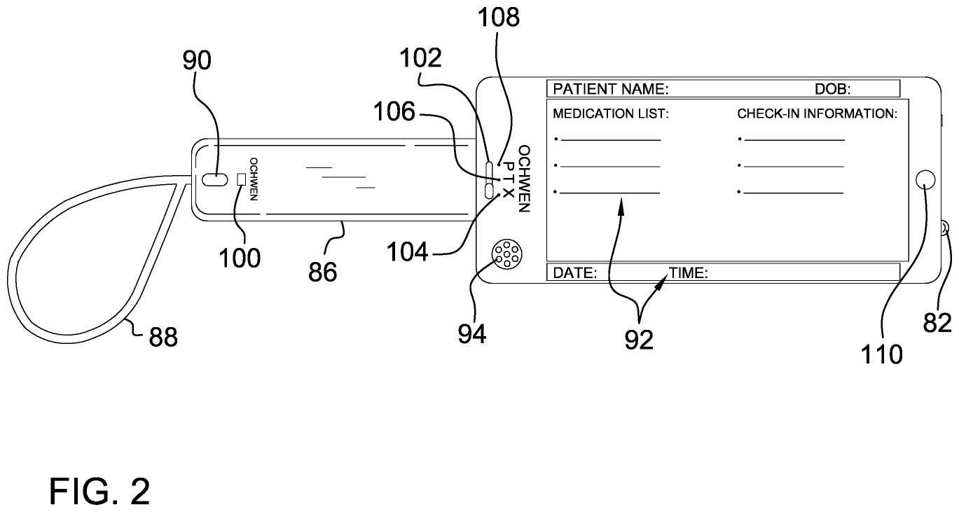

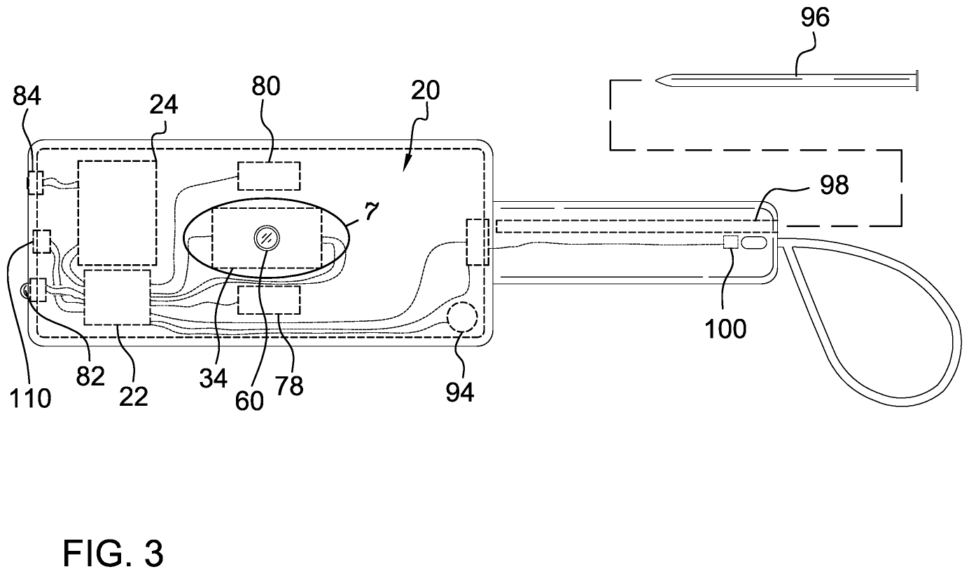

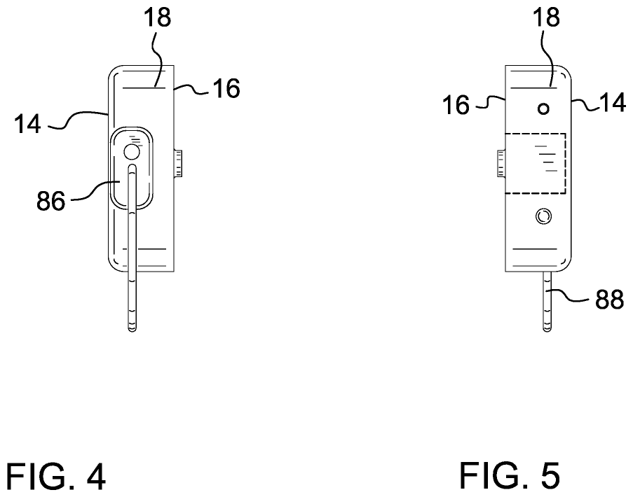

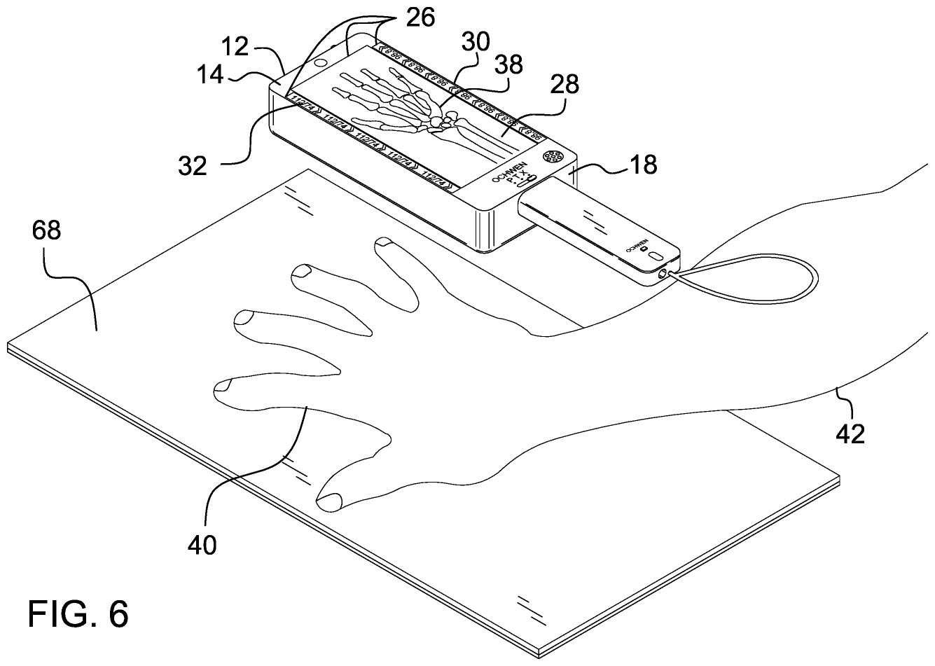

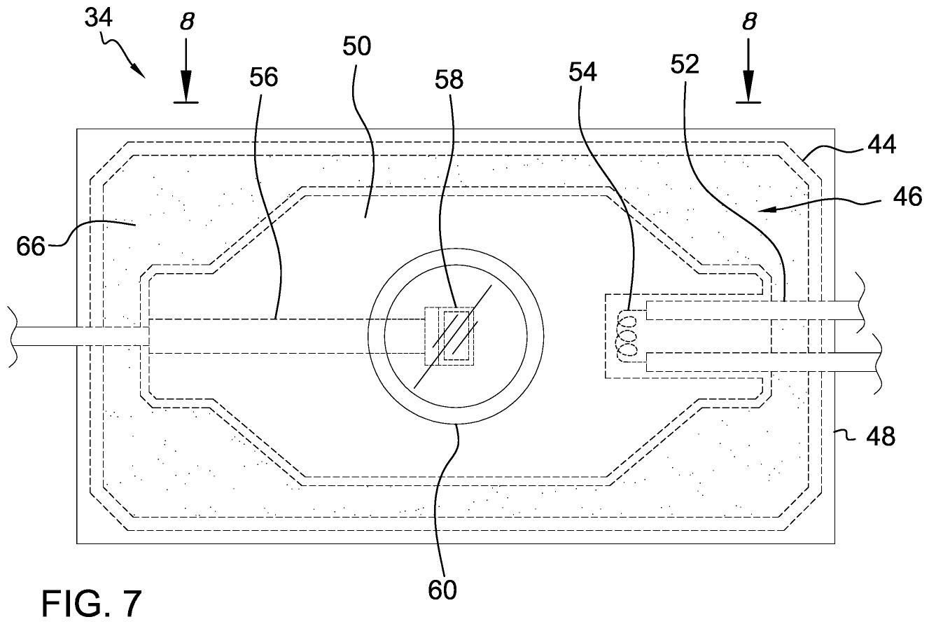

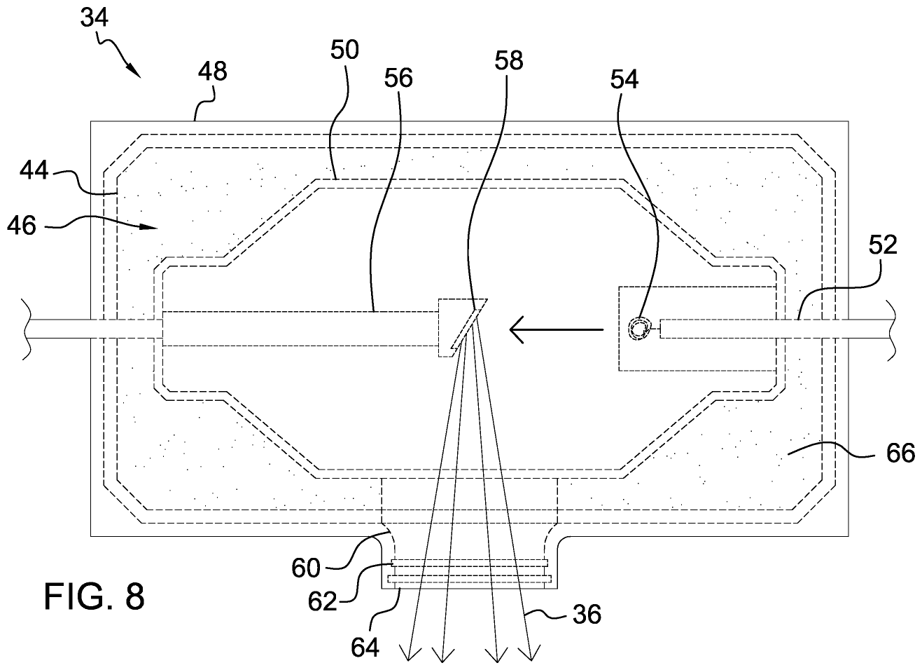

With reference now to the drawings, and in particular to FIGS. 1 through 8 thereof, a new portable medical scanner embodying the principles and concepts of an embodiment of the disclosure and generally designated by the reference numeral 10 will be described.

As best illustrated in FIGS. 1 through 8, the portable medical diagnostic device 10 generally comprises a housing 12 having a top wall 14, a bottom wall 16, and a peripheral wall 18 that is coupled to and extends between the top wall 14 and the bottom wall 16 to define an interior space 20.

A central processing unit 22 is positioned in the interior space 20. The central processing unit 22 may be configured to wirelessly communicate with an external computer network. For example, the external computer network may comprise a wireless internet network or a computer network of a medical facility. The central processing unit 22 may be configured to send and receive data while wirelessly communicating with the external computer network.

A power source 24 is positioned in the interior space 20. The power source 24 is electrically coupled to the central processing unit 22. The power source 24 may be a rechargeable battery.

A display screen 26 is coupled to the housing 12 and is electrically coupled to the central processing unit 22. For example, the display screen 26 may be positioned on the top wall 14. The display screen 26 generally includes an x-ray display section 28, a temperature display section 30, and a blood pressure display section 32. In embodiments, the display screen 26 may comprise a touch screen.

An x-ray scanner 34 is coupled to the housing 12. The x-ray scanner 34 is electrically coupled to the central processing unit 22. The x-ray scanner 34 is configured to emit a plurality of x-ray waves 36 outwardly through the bottom wall 16 to produce an x-ray image 38 of a body part 40 of a user 42. The central processing unit 22 is configured to display the x-ray image 38 on the x-ray display section 28 of the display screen 26.

The x-ray scanner 34 may include a case 44 that is positioned in the interior space 20. The case 44 has an inner area 46. The case 44 may comprise a lead material or another high-Z-material that absorbs x-ray waves 36. As used in this description, a high-Z-material is a chemical element with a high atomic number (Z) of protons in the nucleus. High-Z-materials have highly concentrated electrons and are effective at shielding x-ray waves.

A shield 48 may be coupled to an exterior surface of the case 44. The shield 48 may cover the exterior surface and may comprise a metal material such as a high-Z-material for absorbing stray x-ray waves to inhibit those stray x-ray waves from being emitted out of the case 44.

An x-ray tube 50 is positioned within the inner area 46 of the case 44. The x-ray tube 50 may be under a high vacuum pressure to facilitate acceleration of the electrons passing from a cathode 52 to an anode 56 within the x-ray tube 50. In some embodiments, the x-ray tube 50 may comprise a metal material or a glass material.

The cathode 52 is positioned within the x-ray tube 50. The cathode 52 is electrically coupled to the power source 24. The cathode 52 includes a heated filament 54. The power source 22 passes electric current through the heated filament 54 thereby releasing electrons from the heated filament 54.

The anode 56 is positioned within the x-ray tube 50. The anode 56 is aligned with the cathode 52. The anode 56 draws the electrons across the x-ray tube 50. The electrons collide with the anode 56 forming the plurality of x-ray waves 36. The anode 56 may have an angled surface 58 for directing the plurality of x-ray waves 36 radiating outwardly from the anode 56.

A window 60 extends through the case 44, the shield 48, the x-ray tube 50, and the housing 12. For example, the window 60 may be inset in the bottom wall 16 and exposed within the bottom wall 16. The window 60 may be aligned with the angled surface 58 of the anode 56. The plurality of x-ray waves 36 are permitted to escape outwardly from the housing 12 through the window 60.

A filter 62 may be coupled to the window 60. The filter 62 is generally positioned between an outer surface 64 of the window 60 and the x-ray tube 50. The filter 62 is configured to reduce radiation exposure to the user 42. For example, the filter 62 may comprise one of an aluminum material and a copper material that can absorb low-energy photons that would otherwise be absorbed by the user's soft tissues. Alternatively, the filter 62 may be configured to enhance contrast in the x-ray image 38. For example, the filter 62 may comprise a molybdenum material that can remove some of the high energy photons to improve the contrast of the x-ray image 38.

An oil bath 66 may be positioned in the inner area 46. The oil bath 66 generally surrounds the x-ray tube 50. The oil bath 66 absorbs heat generated by the cathode 52 and the anode 56. The shield 48 and oil bath 66 can insulate the x-ray tube 50 by absorbing the stray x-ray waves, or in other words by inhibiting the plurality of x-ray waves 36 from radiating outwardly from the case 44 except through the window 60.

The x-ray scanner 34 further includes a mat 68, which may have a first layer 70 and a second layer 72. The first layer 70 is generally positioned on top of the second layer 72 wherein the first layer 70 is configured to receive the body part 40 of the user 42. The second layer 72 may comprise a lead material or another high-Z-material that is configured to block the plurality of x-ray waves 36 from passing through the second layer 72. The mat 68 thus inhibits the x-ray scanner 34 from including any extraneous information in the x-ray image 38, such as rocks, metal, or other objects on the surface where the body part 40 of the user 42 is positioned.

The mat 68 may further comprise a plurality of sensors 74 that is embedded within the mat 68. The plurality of sensors 74 is configured to detect the plurality of x-ray waves 36 passing through the user 42 to reach the mat 68.

A transmitter 76 may be embedded within the mat 68. The transmitter 76 is in communication with the plurality of sensors 74. The transmitter 76 is in wireless communication with the central processing unit 22. The transmitter 76 wirelessly communicates the plurality of x-ray waves 36 being detected by the plurality of sensors 74 to the central processing unit 22. The central processing unit 22 is configured to construct the x-ray image 38 from the plurality of x-ray waves 36 being detected by the plurality of sensors 74. The x-ray image 38 is then ready for display on the x-ray display section 28 of the display screen 26.

A temperature scanner 78 is positioned in the interior space 20. The temperature scanner 78 is electrically coupled to the central processing unit 22. The temperature scanner 78 is configured to detect a temperature of the user 42. The central processing unit 22 is configured to display the temperature on the temperature display section 30 of the display screen 26. The temperature may be displayed in a first color of text, such as a green text, when the temperature is within a healthy range, for example between 97.0° Fahrenheit and 99.6° Fahrenheit. The temperature may be displayed in a second color of text, such as a red text, when the temperature not within the healthy range, for example above 100.0° Fahrenheit.

A blood pressure scanner 80 is positioned in the interior space 50. The blood pressure scanner 80 is electrically coupled to the central processing unit 22. The blood pressure scanner 80 is configured to detect a blood pressure of the user 42. The central processing unit 22 is configured to display the blood pressure on the blood pressure display section 32 of the display screen 26. The blood pressure may be displayed in the first color of text when the blood pressure is in a normal range. For example, the normal range may include a systolic pressure being less than 120.0 millimeters of mercury and a diastolic pressure being less than 80.0 millimeters of mercury. The blood pressure may be displayed in the second color of text when the blood pressure is above the normal range.

A flashlight 82 may be electrically coupled to the central processing unit 22. The central processing unit 22 can actuate the flashlight 82. The flashlight 82 may be inset into the peripheral wall 18 and exposed within the peripheral wall 18. The flashlight 82 may have a low-light setting, a medium-light setting, and a high-light setting.

A charging port 84 may be electrically coupled to the power source 24. The charging port 84 may be inset into the peripheral wall 18 and exposed within the peripheral wall 18.

A handle 86 may be coupled to the housing 12. For example, the handle 86 may extend outwardly from the peripheral wall 18. A lanyard 88 may be coupled to the handle 86 distal to the housing 12. A hole 90 may extend through the handle 86. The hole 90 is generally configured to receive a hook or other hanger for hanging the housing 12 when the housing 12 is in storage.

The display screen 26 may be configured to display a piece of information 92 about the user 42. For example, the piece of information 92 may include one or more of a name, a date of birth, a list of medications, a check-in information, a medical history data, a time, and a date.

A speaker 94 may be electrically coupled to the central processing unit 22. The central processing unit 22 may be configured to read the piece of information 92 audibly through the speaker 94.

A stylus 96 may be wirelessly coupled to the central processing unit 22. The stylus 96 is generally configured to facilitate the user 42 in interacting with a user interface display of the display screen 26. A cavity 98 may be positioned within the handle 86 for storing the stylus 96.

An options button 100 may be coupled to the handle 86. The options button 100 is electrically coupled to the central processing unit 22. The central processing unit 22 may be configured to adjust a view of the display screen 26 or otherwise facilitate the user 42 in interacting with the user interface display of the display screen 26 when the options button 100 is manipulated. For example, the view of the display screen 26 may include one or more of the x-ray image 36, the temperature, the blood pressure, and the piece of information 92.

A mode switch 102 may be coupled to the housing 12. The mode switch 102 is electrically coupled to the central processing unit 22 for selectively actuating the x-ray scanner 34, the temperature scanner 78, and the blood pressure scanner 80. For example, the mode switch 102 may be positioned proximate to the handle 86.

The mode switch 102 may include an x-ray position 104. The central processing unit 22 may be configured to actuate the x-ray scanner 34 when the mode switch 102 is in the x-ray position 104. The mode switch 102 may also include a temperature position 106 wherein the central processing unit 22 is configured to actuate the temperature scanner 78 when the mode switch 102 is in the temperature position 106. The mode switch 102 may also include a blood pressure position 108 wherein the central processing unit 22 is configured to actuate the blood pressure scanner 80 when the mode switch 102 is in the blood pressure position 108.

A control switch 110 may be coupled to the housing 12. The control switch 110 is electrically coupled to the central processing unit 22. The control switch 110 selectively actuates one of the x-ray scanner 34, the temperature scanner 78, and the blood pressure scanner 80, for example when the control switch 110 is actuated and while the mode switch 102 is positioned in a respective one of the x-ray position 104, the temperature position 106, and the blood pressure position 108. In embodiments, the x-ray scanner 34 may be actuated to produce the x-ray image 38 when the control switch 110 is actuated while the mode switch 102 is in the x-ray position 104. The temperature scanner 78 may detect the temperature when control switch 110 is actuated while the mode switch 102 is in the temperature position 106. The blood pressure scanner 80 may detect the blood pressure when the control switch 110 is actuated while the mode switch 102 is in the blood pressure position 108.

In use, the user 42 can position the body part 40 for which the user 42 wishes to generate the x-ray image 38 on the mat 68. The user 42 can move the mode switch 102 to the x-ray position 104 and actuate the control switch 110 to generate the plurality of x-ray waves 36 needed for the central processing unit 22 to create the x-ray image 38. The user 42 can move the mode switch 102 to the temperature position 106 and actuate the control switch 110 to obtain the temperature reading. The user 42 can move the mode switch 102 to the blood pressure position 108 and actuate the control switch 110 to obtain the blood pressure reading. All the diagnostic information can be conveniently displayed on the display screen 26. A user 42, or a medical professional, can also view the piece of information 92 related to the user 42, or the patient, on the display screen 26. Medical personnel can use the single portable medical diagnostic device 10 to obtain vital diagnostic information about a patient without needing to use multiple rooms or wait in lines for use of multiple machines.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A medical scanner system comprising:
a housing having a top wall, a bottom wall, and a peripheral wall being coupled to and extending between the top wall and the bottom wall to define an interior space;
a central processing unit being positioned in the interior space;
a power source being positioned in the interior space, the power source being electrically coupled to the central processing unit;
a display screen being coupled to the housing and being electrically coupled to the central processing unit, the display screen including an x-ray display section, a temperature display section, and a blood pressure display section;
an x-ray scanner being coupled to the housing, the x-ray scanner being electrically coupled to the central processing unit wherein the x-ray scanner is configured to emit a plurality of x-ray waves outwardly through the housing to produce an x-ray image of a body part of a user, the central processing unit being configured to display the x-ray image on the x-ray display section of the display screen;
a temperature scanner being positioned in the interior space, the temperature scanner being electrically coupled to the central processing unit, the temperature scanner being configured to detect a temperature of the user, the central processing unit being configured to display the temperature on the temperature display section of the display screen;
a blood pressure scanner being positioned in the interior space, the blood pressure scanner being electrically coupled to the central processing unit, the blood pressure scanner being configured to detect a blood pressure of the user, the central processing unit being configured to display the blood pressure on the blood pressure display section of the display screen.

2. The medical scanner system of claim 1, the x-ray scanner further comprising:
a case being positioned in the interior space, the case having an inner area;
an x-ray tube being positioned within the inner area;
a cathode being positioned within the x-ray tube, the cathode being electrically coupled to the power source, the cathode including a heated filament wherein the power source passes electric current through the heated filament thereby releasing electrons from the heated filament;
an anode being positioned within the x-ray tube, the anode being aligned with the cathode wherein the anode draws the electrons across the x-ray tube and wherein the electrons collide with the anode forming the plurality of x-ray waves;
a window extending through the case, the x-ray tube, and the housing, the window being inset in the bottom wall and being exposed within the bottom wall wherein the plurality of x-ray waves are permitted to escape outwardly from the housing through the window;
a mat comprising a lead material being configured to block the plurality of x-ray waves from passing through the mat, the mat further comprising:
a plurality of sensors being embedded within the mat, the plurality of sensors being configured to detect the plurality of x-ray waves passing through the user to reach the mat; and
a transmitter being embedded within the mat, the transmitter being in communication with the plurality of sensors, the transmitter being in wireless communication with the central processing unit, the transmitter wirelessly communicating the plurality of x-ray waves being detected by the plurality of sensors to the central processing unit wherein the central processing unit is configured to construct the x-ray image from the plurality of x-ray waves being detected by the plurality of sensors.

3. The medical scanner system of claim 2, further comprising a shield being coupled to an exterior surface of the case, the shield covering the exterior surface, wherein the window extends through the shield.

4. The medical scanner system of claim 2, further comprising a filter being coupled to the window, the filter being positioned between an outer surface of the window and the x-ray tube, the filter being configured to reduce radiation exposure to the user.

5. The medical scanner system of claim 2, further comprising an oil bath being positioned in the inner area, the oil bath surrounding the x-ray tube, the oil bath absorbing heat generated by the cathode and the anode.

6. The medical scanner system of claim 1, wherein the temperature is displayed in a green text when the temperature is between 97.0° Fahrenheit and 99.0° Fahrenheit.

7. The medical scanner system of claim 1, wherein the blood pressure is displayed in a green text when the blood pressure is in a normal range, the normal range including a systolic pressure being less than 120.0 millimeters of mercury and a diastolic pressure being less than 80.0 millimeters of mercury.

8. The medical scanner system of claim 1, wherein the display screen is configured to display a piece of information about the user, the piece of information including one or more of a name, a date of birth, a list of medications, a check-in information, a medical history data, a time, and a date.

9. The medical scanner system of claim 8, further comprising a speaker being electrically coupled to the central processing unit, the central processing unit being configured to read the piece of information audibly through the speaker.

10. The medical scanner system of claim 1, further comprising:
a stylus being wirelessly coupled to the central processing unit, the stylus being configured to facilitate the user in interacting with a user interface display of the display screen; and
a cavity being positioned within the handle for storing the stylus.

11. The medical scanner system of claim 1, further comprising an options button being coupled to the handle, the options button being electrically coupled to the central processing unit, wherein the central processing unit is configured to adjust a view of the display screen when the options button is manipulated.

12. The medical scanner system of claim 1, further comprising a mode switch being coupled to the housing, the mode switch being electrically coupled to the central processing unit for selectively actuating the x-ray scanner, the temperature scanner, and the blood pressure scanner.

13. The medical scanner system of claim 12, the mode switch further comprising:
an x-ray position wherein the central processing unit is configured to actuate the x-ray scanner when the mode switch is in the x-ray position;
a temperature position wherein the central processing unit is configured to actuate the temperature scanner when the mode switch is in the temperature position; and
a blood pressure position wherein the central processing unit is configured to actuate the blood pressure scanner when the mode switch is in the blood pressure position.

14. The medical scanner system of claim 1, further comprising a control switch being coupled to the housing, the control switch being electrically coupled to the central processing unit, the control switch selectively actuating one of the x-ray scanner, the temperature scanner, and the blood pressure scanner when the control switch is actuated.

15. The medical scanner system of claim 1, further comprising a flashlight being electrically coupled to the central processing unit, the flashlight being inset into the peripheral wall and being exposed within the peripheral wall.

16. The medical scanner system of claim 1, the flashlight further comprising a low-light setting, a medium-light setting, and a high-light setting.

17. The medical scanner system of claim 1, further comprising a charging port being electrically coupled to the power source.

18. The medical scanner system of claim 1, wherein the central processing unit is configured to wirelessly communicate with an external computer network

19. A medical scanner system comprising:
a housing having a top wall, a bottom wall, and a peripheral wall being coupled to and extending between the top wall and the bottom wall to define an interior space;
a central processing unit being positioned in the interior space, the central processing unit being configured to wirelessly communicate with an external computer network;
a power source being positioned in the interior space, the power source being electrically coupled to the central processing unit, the power source being a rechargeable battery;
a display screen being coupled to the housing and being electrically coupled to the central processing unit, the display screen being positioned on the top wall, the display screen including an x-ray display section, a temperature display section, and a blood pressure display section, the display screen being a touch screen;
an x-ray scanner being coupled to the housing, the x-ray scanner being electrically coupled to the central processing unit wherein the x-ray scanner is configured to emit a plurality of x-ray waves outwardly through the bottom wall to produce an x-ray image of a body part of a user, the central processing unit being configured to display the x-ray image on the x-ray display section of the display screen, the x-ray scanner including:
a case being positioned in the interior space, the case having an inner area, the case comprising a lead material;
a shield being coupled to an exterior surface of the case, the shield covering the exterior surface, the shield comprising a metal material;
an x-ray tube being positioned within the inner area;
a cathode being positioned within the x-ray tube, the cathode being electrically coupled to the power source, the cathode including a heated filament wherein the power source passes electric current through the heated filament thereby releasing electrons from the heated filament;
an anode being positioned within the x-ray tube, the anode being aligned with the cathode wherein the anode draws the electrons across the x-ray tube and wherein the electrons collide with the anode forming the plurality of x-ray waves, the anode having an angled surface directing the plurality of x-ray waves radiating outwardly from the anode;
a window extending through the case, the shield, the x-ray tube, and the housing, the window being inset in the bottom wall and being exposed within the bottom wall, the window being aligned with the angled surface of the anode wherein the plurality of x-ray waves are permitted to escape outwardly from the housing through the window;
a filter being coupled to the window, the filter being positioned between an outer surface of the window and the x-ray tube, the filter being configured to reduce radiation exposure to the user, the filter comprising one of an aluminum material and a copper material;
an oil bath being positioned in the inner area, the oil bath surrounding the x-ray tube, the oil bath absorbing heat generated by the cathode and the anode;
a mat having a first layer and a second layer, the first layer being positioned on top of the second layer wherein the first layer is configured to receive the body part of the user, the second layer comprising a lead material being configured to block the plurality of x-ray waves from passing through the second layer, the mat further comprising:
a plurality of sensors being embedded within the mat, the plurality of sensors being configured to detect the plurality of x-ray waves passing through the user to reach the mat;
a transmitter being embedded within the mat, the transmitter being in communication with the plurality of sensors, the transmitter being in wireless communication with the central processing unit, the transmitter wirelessly communicating the plurality of x-ray waves being detected by the plurality of sensors to the central processing unit wherein the central processing unit is configured to construct the x-ray image from the plurality of x-ray waves being detected by the plurality of sensors, the x-ray image being displayed on the x-ray display section of the display screen;
a temperature scanner being positioned in the interior space, the temperature scanner being electrically coupled to the central processing unit, the temperature scanner being configured to detect a temperature of the user, the central processing unit being configured to display the temperature on the temperature display section of the display screen, wherein the temperature is displayed in a first color of text when the temperature is between 97.0° Fahrenheit and 99.6° Fahrenheit and wherein the temperature is displayed in a second color of text when the temperature is above 100.0° Fahrenheit;
a blood pressure scanner being positioned in the interior space, the blood pressure scanner being electrically coupled to the central processing unit, the blood pressure scanner being configured to detect a blood pressure of the user, the central processing unit being configured to display the blood pressure on the blood pressure display section of the display screen, wherein the blood pressure is displayed in the first color of text when the blood pressure is in a normal range, the normal range including a systolic pressure being less than 120.0 millimeters of mercury and a diastolic pressure being less than 80.0 millimeters of mercury, wherein the blood pressure is displayed in the second color of text when the blood pressure is above the normal range;
a flashlight being electrically coupled to the central processing unit, the central processing unit actuating the flashlight, the flashlight being inset into the peripheral wall and being exposed within the peripheral wall, the flashlight having a low-light setting, a medium-light setting, and a high-light setting;
a charging port being electrically coupled to the power source, the charging port being inset into the peripheral wall and being exposed within the peripheral wall;
a handle being coupled to the housing, the handle extending outwardly from the peripheral wall;
a lanyard being coupled to the handle distal to the housing;
a hole extending through the handle, the hole being configured to receive a hook for hanging the housing when the housing is in storage;
wherein the display screen is configured to display a piece of information about the user, the piece of information including one or more of a name, a date of birth, a list of medications, a check-in information, a medical history data, a time, and a date;
a speaker being electrically coupled to the central processing unit, the central processing unit being configured to read the piece of information audibly through the speaker;
a stylus being wirelessly coupled to the central processing unit, the stylus being configured to facilitate the user in interacting with a user interface display of the display screen;
a cavity being positioned within the handle for storing the stylus;
an options button being coupled to the handle, the options button being electrically coupled to the central processing unit, wherein the central processing unit is configured to adjust a view of the display screen when the options button is manipulated, the view including one or more of the x-ray image, the temperature, the blood pressure, and the piece of information;
a mode switch being coupled to the housing, the mode switch being electrically coupled to the central processing unit for selectively actuating the x-ray scanner, the temperature scanner, and the blood pressure scanner, the mode switch being positioned proximate to the handle, the mode switch including:
an x-ray position wherein the central processing unit is configured to actuate the x-ray scanner when the mode switch is in the x-ray position;
a temperature position wherein the central processing unit is configured to actuate the temperature scanner when the mode switch is in the temperature position;
a blood pressure position wherein the central processing unit is configured to actuate the blood pressure scanner when the mode switch is in the blood pressure position; and
a control switch being coupled to the housing, the control switch being electrically coupled to the central processing unit, the control switch selectively actuating one of the x-ray scanner, the temperature scanner, and the blood pressure scanner when the control switch is actuated and when the mode switch is positioned in one of the x-ray position, the temperature position, and the blood pressure position, wherein the x-ray scanner produces the x-ray image when the control switch is actuated while the mode switch is in the x-ray position, wherein the temperature scanner detects the temperature when control switch is actuated while the mode switch is in the temperature position, and wherein the blood pressure scanner detects the blood pressure when the control switch is actuated while the mode switch is in the blood pressure position.
